(19) United States
(12) Patent Application Publication
(10) Pub. No.: US 2006/0034908 A1
(43) Pub. Date: Feb. 16, 2006

(54) Manufacturing process for liposomal preparations

(75) Inventors: Shastri Bhamidipati, Buffalo Grove, IL (US); Zafeer Ahmad, Wadsworth, IL (US); Imran Ahmad, Wadsworth, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NeoPharm, Inc., Lake Forest, IL (US)

(21) Appl. No.: 11/201,810

(22) Filed: Aug. 11, 2005

Related U.S. Application Data
(63) Continuation of application No. PCT/US04/04555, filed on Feb. 11, 2004.
(60) Provisional application No. 60/446,895, filed on Feb. 11, 2003.

(51) Int. Cl. A61K 9/127

(52) U.S. Cl. 424/450

(57) ABSTRACT

The present invention provides manufacturing processes for liposomal preparations. In accordance with the methods, a lipid fraction is dissolved in a water-miscible organic solvent. This solution comprising the lipid fraction can be added to and mixed with an aqueous solution under conditions to form a bulk liposomal preparation. Desirably, the preparation can include one or more active principals. The bulk liposomal preparation can be further processed as desired, for example, by size fractionation or reduction, removal of the water-miscible organic solvent, freeze-drying, or other treatment. The methods permit the production of liposomal formulations on a large or commercial scale.

### CROSS-REFERENCE TO RELATED APPLICATIONS

This application is a continuation of PCT/US04/04555 filed on Feb. 11, 2004, which claims priority to co-pending U.S. Provisional Patent Application 60/446,895, filed on Feb. 11, 2003. The disclosures of these applications are incorporated herein in their entireties by reference thereto.

### TECHNICAL FIELD OF THE INVENTION

The present invention relates to methods of manufacturing a liposomal preparation and the liposomal preparation produced by these methods.

### BACKGROUND OF THE INVENTION

Many known methods exist for manufacturing liposomal formulations of various active principals (typically antineoplastic agents, antifungal agents, and the like). Such methods include, for example, ethanol dilution, thin film hydration, the methylene chloride process, and the like.

Other methods have been proposed involving t-butanol to dissolve liposome-forming-lipids to manufacture dried lipid powders by lyophilization (see, e.g., U.S. Pat. Nos. 6,146,659 and 6,090,407). Hydration of the dried lipid powders with suitable aqueous media results in the formation of multi-lamellar liposomes that are size reduced by sonication and nebulization for administration. T-butanol has not been used as the primary choice solvent for manufacturing liposomes, however, mainly due to: i) its limited lipid solubility (cholesterol in particular) in t-butanol; ii) its acceptability as a pharmaceutical excipient in parenteral dosage forms; iii) the necessity for its removal upon liposome formation.

While effective on a small-scale basis, current methods generally are unsuitable for manufacturing large quantities of liposomal formulations of many active principals, particularly paclitaxel and other anticancer agents. As a result, current methods for manufacturing liposomal formulations are not adequate to supply commercial quantities of liposomal preparations of many pharmaceutical agents. Thus, there is a need for a process for manufacturing liposomal formulations of active principals that can be employed on a large or commercial scale.

### SUMMARY OF THE INVENTION

The present invention provides manufacturing processes for liposomal preparations. In accordance with one aspect of the inventive method, a lipid fraction is dissolved in a water-miscible organic solvent. This solution comprising the lipid fraction can be added to and mixed with an aqueous solution under controlled conditions suitable to form a bulk liposomal preparation.

Desirably, the preparation can include one or more active principals. In accordance with another aspect of the inventive method, at least one active principal and a lipid fraction are dissolved in a water-miscible organic solvent. This solution comprising the active principal and lipid fraction can be added to and mixed with an aqueous solution under controlled conditions suitable to form a bulk liposomal preparation.

The bulk liposomal preparation can be further processed as desired, for example by size fractionation or reduction, removal of the water-miscible organic solvent, sterilization by membrane filtration, freeze-drying, or other treatment.

The invention further provides a liposomal preparation produced by the manufacturing processes of the present invention and methods of using such formulations.

The invention permits the production of liposomal formulations on a commercial scale. These advantages of the present invention, and additional inventive features, will be apparent from the following detailed description and the accompanying drawings.

### BRIEF DESCRIPTION OF THE DRAWINGS

FIG. 1 is a histogram presenting the size distribution of paclitaxel containing liposomes prepared by a process utilizing t-butanol after size reduction.

FIG. 2 is a flow chart for solvent removal by tangential flow filtration.

FIG. 3 is a histogram presenting the size distribution of paclitaxel containing liposomes prepared by a process utilizing t-butanol after size reduction and solvent removal by tangential flow-filtration.

FIG. 4 is a histogram presenting the size distribution of paclitaxel containing liposomes (reconstituted after freeze drying) prepared by a process utilizing t-butanol.

FIG. 5 is a freeze fracture electron micrograph of paclitaxel containing liposomes (reconstituted after freeze drying) prepared by a process utilizing t-butanol.

### DETAILED DESCRIPTION OF THE INVENTION

In accordance with the inventive methods, a water-miscible organic solvent is employed to dissolve a lipid fraction and/or one or more active principals. Many such water-miscible organic solvents (e.g. dimethylsulfoxide, ethanol, and methanol) can be used in the context of the present invention. However, the most preferred water-miscible organic solvent is t-butanol.

The lipid fraction can comprise any suitable lipid or lipids of which it is desired to form liposomes. Preferred lipids in the lipid fraction include, for example, one or more of cholesterol, dioleoylphosphatidylcholine (DOPC), tetramyristoyl cardiolipin, and tocopheryl acid succinate. In some embodiments, tetramyristoyl cardiolipin can be substituted with positively charged cationic cardiolipins, such as 1,3-Bis-(1,2-bis-tetradecyloxy-propyl-3-dimethylethoxyammoniumbromide)-propan-2-ol [(R)-PCL-2] and the like. Preferably, the lipid fraction includes an antioxidant, such as tocopheryl acid succinate. More preferably, the lipid fraction includes at least two (such as three or more) of these compounds, and most preferably the lipid fraction includes this entire group of compounds. Depending on the desired composition of the lipid fraction, the amount of the various lipids can be adjusted as desired. However, a preferred composition of the lipid fraction includes a majority of the lipids as DOPC, for example a DOPC:Chol:Cardiolipin 90:5:5 molar ratio. Where an antioxidant is included, a suitable molar ratio is 89:5:5:1 DOPC:Chol:Cardiolipin:Tocopheryl acid succinate.

In some embodiments, an effective formulation can be produced by sequential addition or dissolution of the lipids that form the lipid fraction in the water-miscible organic solvent. Most preferably, the method involves sequential addition of cholesterol, DOPC, tetramyristoyl cardiolipin, and tocopheryl acid succinate so as to dissolve each into the water-miscible organic solvent. In many embodiments, it is desirable for the lipid fraction to be dissolved in the water-miscible organic solvent at temperatures above room temperature (i.e., about 25° C.). Thus, where t-butanol is the desired solvent, the lipids can be added at temperatures between about 35° C. and about 65° C., such as between about 45° C. and about 55° C.

After the lipid fraction is added to the water-miscible organic solvent, the resulting solution can be added to an aqueous solution to form a bulk liposome preparation. At this stage, the bulk liposome preparation typically comprises multilamellar liposomes, as assessed, for example, by dynamic light scattering.

For forming the bulk liposome preparation, the amount of aqueous solution can vary, but generally it is a majority of the batch size, e.g., the volume of the total liposome preparation. Preferably, the amount of aqueous solution is at least about 80% of batch size, and the amount of aqueous solution more preferably is at least about 90% of batch size. In some embodiments, the amount of aqueous solution can be more than the batch size.

The aqueous solution can be water but more typically contains one or more additional ingredients, such as sugars, tonicity adjusters, and the like. Suitable tonicity adjusters include salts (preferably sodium chloride) and other agents known to those of ordinary skill in the art. Tonicity adjusters can be present in any suitable amount; however, when present, the tonicity adjusters typically represent less than about 2% of the aqueous solution, and more typically less than about 1% of the aqueous solution. Preferably, the aqueous solution contains a protective sugar (such as, for example, trehalose, sucrose, maltose, lactose, glucose, dextran, etc., as well as combinations of these). One or more of such protective sugars can be present in any suitable amount. However, when present, the protective sugar(s) adjusters typically represent at least about 5% of the solution, and generally less than about 20% of the aqueous solution (more typically less than about 15% of the aqueous solution). A most preferred aqueous solution for this purpose is 10-12% sucrose and 0.4-0.9% sodium chloride.

The water-miscible organic solvent solution containing the lipid fraction can be added to the aqueous solution by any method able to achieve the formation of the bulk liposome preparation. However, allowing the lipid solution in t-butanol to cool below 40° C. results in lipid precipitation. Likewise, addition of lipid solution to aqueous phase solution maintained at room temperature also can result in precipitation of lipid and (when present) active principal. Accordingly, it is preferable for the water-miscible organic solvent solution to be added to the aqueous solution with mixing (e.g., using a conventional mixer, such as those manufactured by Labmaster), for example at between about 300 rpm to about 400 rpm, while maintaining the temperature above 30° C., such as maintaining the aqueous solution at between about 30° C. and about 40° C. Also, it often will assist the formation of liposomes for the water-miscible organic solvent solution containing the liposomal fraction to be added to the aqueous solution while maintaining the temperature at about 35° C. For example, when added to the aqueous solution, the water-miscible organic solvent can be maintained between about 25° C. and about 40° C., more preferably between about 30° C. and about 40° C., and most preferably between about 30° C. and about 35° C., particularly where the water-miscible organic solvent is t-butanol.

The rate at which the water miscible organic solvent containing lipid fraction is added to the aqueous solution and the rate of mixing of aqueous solution during such addition manifest the formation of liposomes containing the lipid soluble active principal (paclitaxel, docetaxel) without precipitation. For example, where t-butanol serves as the water-miscible organic solvent, it and the aqueous solutions can be combined while mixing for between about 5 minutes and about 1 hour, more typically between about 10 minutes and about 45 minutes, and typically between about 15 minutes and about 30 minutes. For large-scale production, the duration of addition (e.g., period of mixing) can be considerably longer, such as several hours or more. Also, the mixing speed can be somewhat less than 300 rpm or somewhat more than 400 rpm, as noted above, as needed, such as, for example, at least about 200 rpm or at least about 500 rpm and up to about 800 rpm or even up to about 1000 rpm. Thus, the mixing speed can be between about 200 rpm and about 800 rpm, such as between about 500 rpm and about 1000 rpm. For large scale production, a preferred range is between about 600 rpm and about 800 rpm.

Alternatively, the addition of the water-miscible organic solvent solution comprising the lipid fraction to the aqueous solution can be accomplished while the solution is cooling. Typically, this involves mixing of solution following addition of water-miscible solvent comprising the lipid fraction to the aqueous solution while cooling. For example, the water-miscible organic solvent solution with the lipid fraction can be added to the aqueous solution while cooling to a temperature between about 25° C. and about 30° C.

In many applications it is desirable for the liposomal preparation to be used in medical applications. For such applications, the preparation can contain one or more active principals. An active principal can be any agent (or combination of agents) desired to be formulated into a liposomal preparation, such as a small molecule, oligonucleotide, or other agent. Typically, the active principal includes at least one antineoplastic or antifungal agent. Preferred active principals are agents such as taxanes or derivatives, such as paclitaxel, docetaxel, and related compounds (e.g., epothilones A and B, epothilone derivatives, etc.) and other anticancer agents such mitoxantrone, camptothecins, and related molecules (such as, for example, 7-ethyl-10-hydroxycamptothecin (i.e., SN-38), irinotecan, etc.) and derivatives, doxorubicin, daunorubicin, methotrexate, adriamycin, tamoxifen, toremifene, cisplatin, epirubicin, gemcitabicine HCl, mixotantrone, and other known chemotherapeutics useful for treatment of cancer and antisense oligonucleotides (such as antisense oligonucleotides that inhibit the expression of an oncogene, see, e.g., U.S. Pat. Nos. 6,559,129, 6,333,314, and 6,126,965, disclosing a 15-mer anti c-raf-1 oligonucleotide having the sequence 5′-GTGCTCCATTGATGC-3′).

Preferably the active principal comprises at least one agent selected from the group consisting of taxanes or derivatives and camptothecin or derivatives. One of ordinary skill in the art will recognize that derivatives or analogs will have the same activity as the unaltered agent, optionally to a greater or lesser extent, but not negated. Such chemical modifications will be based on structure activity relationships (SAR) or molecular modeling. For example, functional groups can be substituted or eliminated. A most preferred active principal is paclitaxel.

While any amount of active principal can be employed, as desired, where paclitaxel is used, typically an amount of active principal of at least about 1% weight, relative to the batch size, is dissolved in the water-miscible organic solvent. More typically, at least where 1 mg/ml paclitaxel (relative to batch size) is employed, the paclitaxel is dissolved in at least about 5% by volume of t-butanol, relative to batch size. It is possible, in some embodiments, for the amount of active principal to exceed about 5% by volume, relative to batch size. In the same manner, up to 10% by volume t-butanol or a mixture of t-butanol and ethanol not exceeding 1:1 (volume ratio) and a total of 10% by volume may be used.

The one or more active principals are added during the formulation process in a manner appropriate to the chemistry of the compound. For example, water-soluble principals (e.g., antisense oligonucleotides) can be added to the aqueous solution, such as before bulk liposome formation. The addition of the water-soluble principals to the aqueous solution can be prior to the addition of the water-miscible organic solvent so as to be entrapped in the liposomes or bound to the liposomes. Alternatively, some water-soluble active principals (e.g., SN38) can be added to the size reduced liposomes after solvent removal but before sterile filtration and freeze-drying, steps which are described below. Further, active principals, such as ones that are soluble in organic solvents, can be added by dissolving them in the water-miscible organic solvent. Preferably, the active principals soluble in organic solvents can be added to the water-miscible organic solvent prior to the addition of the lipid fraction. The one or more active principals can be added during or after mixing the water-miscible organic solvent solution comprising the lipid fraction with the aqueous solution.

In many embodiments, it is desirable for the one or more active principals (excluding water soluble agents) to be dissolved in the water-miscible organic solvent, particularly t-butanol, at temperatures above room temperature (e.g., about 35° C.). Thus, for example, where paclitaxel is the desired active principal, it can be dissolved in a water-miscible organic solvent, such as t-butanol, at temperatures between about 35° C. and about 65° C., such as between about 40° C. and about 55° C. The temperature at which other active principals can be dissolved in t-butanol or in other water-miscible organic solvents may vary depending on the properties of the active principals, but it is within the ordinary skill of the art to select a suitable temperature for dissolution. As mentioned above, it often is desirable for the water-miscible organic solvent solution containing the lipid fraction and the active principal to be added to the aqueous solution while maintaining the temperature.

It is often preferred that the bulk liposome preparation formed by these methods be size reduced or fractionated or otherwise controlled. Such a sizing treatment is preferably applied to render the particle size of the liposomes more uniform. The mean size of the liposome formulation can be, for example, about 50 nm to about 200 nm, preferably 100-180 nm, and more preferably 100-160 nm as measured by dynamic light scattering techniques. In addition, 99 percentile distribution (D99) of the size reduced liposomes can be, for example, about 100 nm to about 400 nm, preferably 150-300 nm, more preferably 180-250 nm as measured by dynamic light scattering techniques. An exemplary way to achieve this is to treat the bulk liposome preparation by extrusion through a sieve, such as a polycarbonate filter, of a pre-selected size (such as 0.2 μm, 0.1 μm, etc.). Preferably, the liposomes are size reduced by extrusion through 0.2 μm and 0.1 μm polycarbonate filters at pressures typically up to about 200 psi without precipitation of any active principal from the preparation. For larger scale production, the pressure can be expanded beyond about 200 psi, such as between about 200 psi and about 800 psi.

The bulk liposome preparation (or the size-reduced preparation) will contain most of the water-miscible organic solvent employed initially to dissolve the lipid fraction. For many applications, principally medical uses, it is desirable to substantially remove the solvent (and more desirably, completely remove the solvent) from the bulk or size-reduced liposome preparation. Furthermore, if the preparation is to be freeze dried, it is essential to substantially remove (preferably completely remove) the water-miscible organic solvent, t-butanol, to preserve liposome size and maintain active principal in the liposomes during the freeze drying process. One preferred method of substantially freeing the liposome preparation from water-miscible organic solvent (particularly t-butanol) involves diafiltration using a tangential flow filtration process.

As an example, size reduced liposomes can be recirculated through nominal molecular weight cut-off (MWCO) (ranging from 10,000 Daltons to 500,000 Daltons) membrane filter cassette or cartridge with surface areas ranging from 0.1 sq. meters to several hundred sq. meters that permit the passage of small molecules with less than 1000 Daltons. Recirculation of liposome solution through these membrane filters and by way of restricting the outlet flow, transmembrane pressure can be generated against the pores in the membrane allowing small molecules (e.g., 10% sugar solution and t-butanol) to pass through. This procedure can be performed either in continuous mode or concentration mode. In continuous mode, aqueous phase used in preparing the liposomes is added to the recirculating liposomes at the same rate as the filtrate is removed. In concentration-dilution mode, aqueous phase containing t-butanol is removed from the size reduced liposomes, thus concentrating the liposome solution to a desired volume, preferably 50% of the initial volume, and then adding aqueous phase used in preparing the liposomes to return back to starting volume. This procedure can be repeated in an iterative manner until the water-miscible solvent (e.g., t-butanol) is removed to desired levels, preferably less than 1% of the total volume. In either continuous or concentration-dilution mode, a minimum of four volumes (initial starting volume) of aqueous phase is exchanged to remove t-butanol to acceptable levels.

Sterile filtration of liposomal products is an alternate to conventional sterilization procedures (terminal heat sterilization such as autoclaving, gamma radiation, and ethylene oxide treatment), which is a prerequisite (regulatory requirement) for all parenteral dosage forms of medicinal application. By way of passing the liposomes through a sterile 0.22μ filter, all viable microbes are removed from the liposome product. Sterile filtration is performed prior to filling the product in sterilized containers under aseptic conditions.

Following production, and (if desired) size-control and/or removal of water-miscible organic solvent, the bulk or size-reduced lipid preparation preferably is freeze-dried. Any suitable device or method can be employed. A preferred device is a Genesis—25EL (manufactured by Virtis) and any suitable size lyophilizer (e.g., such as those manufactured by Virtis, Edwards, and Hull Corp.). The bulk or size-reduced liposome preparation can be maintained in lyophilized form (e.g., in cold storage at about −2-8° C.) for an extended period of time, such as for at least about several months or years.

For use, the lyophilized bulk or size-fractionated liposomal preparation can be reconstituted with a suitable volume of reconstitution solution, which preferably is a polar solvent, and most preferably an aqueous system, which can be de-ionized water or sterile water or a suitable aqueous saline solution. Any suitable volume of reconstitution solution can be employed, such as between about 1 ml and about 50 ml, more typically between about 3 ml and about 25 ml. For use, the liposomal formulation can be diluted as desired, such as in a suitable physiologically-compatible buffer or saline solution. To assist in reconstitution, the preparation can be mixed gently or vigorously agitated (snapping motion using thumb and index finger) as desired.

The invention further provides a liposomal preparation produced by the manufacturing processes as described herein and methods of using such formulations. The inventive liposomal preparation typically can be formulated for administration to a human or animal patient. For such uses, the inventive formulation can include, in addition to liposome formulations of active agents non-toxic, inert pharmaceutically suitable excipients. Pharmaceutically suitable excipients include solid, semi-solid or liquid diluents, fillers and formulation auxiliaries of all kinds. Tablets, dragees, capsules, pills, granules, suppositories, solutions, suspensions and emulsions, pastes, ointments, gels, creams, lotions, powders and sprays can be suitable pharmaceutical preparations. Suppositories can contain, in addition to the liposomal active agent, suitable water-soluble or water-insoluble excipients. Suitable excipients are those in which the inventive liposomal active agent is sufficiently stable to allow for therapeutic use, for example polyethylene glycols, certain fats, and esters or mixtures of these substances. Ointments, pastes, cream, and gels can also contain suitable excipients in which the liposomal active agent is stable. It is within the ordinary skill in the art to formulate liposomal preparations depending on the desired manner of application (e.g., parenterally, topically, orally, etc.)

The invention also includes pharmaceutical preparations in dosage units. This means that the preparations are in the form of individual parts, for example vials, syringes, capsules, pills, suppositories, or ampoules, of which the content of the liposome formulation of active agent corresponds to a fraction or a multiple of an individual dose. The dosage units can contain, for example, 1, 2, 3, or 4 individual doses, or ½, ⅓, or ¼ of an individual dose. An individual dose preferably contains the amount of active agent which is given in one administration and which usually corresponds to a whole, a half, a third, or a quarter of a daily dose.

The inventive formulations, especially those that contain active agents, facilitate a method of treating a disease in a vertebrate (such as a human or non-human animal), comprising the step of administering a pharmaceutical preparation as described herein, which typically includes a therapeutic agent specific for the treatment of the disease, to the patient. In accordance with the inventive method, a preparation as herein described (desirably containing an active agent) is administered to a vertebrate in need of treatment in an amount and at a location sufficient to treat the disease within the vertebrate. The pharmaceutical preparation is administered to the patient in the manner appropriate to the type of formulation, such as intravenously, subcutaneously, locally, topically (e.g., to skin or dermal tissue, or to mucosal tissue), orally, parenterally, intraperitoneally, rectally, by direct injection into tumors or sites in need of treatment, etc. by such methods as are known or developed.

In one embodiment, the method disease is cancer, in which instance, the pharmaceutical preparation can comprise a suitable anticancer agent, such as herein described. In another embodiment, the disease is an infection, such as a viral, bacterial, or fungal infection. It should be realized that the effective treatment of a disease, in accordance with the inventive methods, while desirably eliminates the disease or its symptoms, need not completely eradicate the effects of the disease. Indeed, successful therapy in accordance with the inventive method can be measured by a reduction in the severity of a disease, infection, or a reduction in the rate by which a disease progresses within a patient. The following examples further illustrate the invention but, of course, should not be construed as in any way limiting its scope.

### EXAMPLE

The example demonstrates the manufacturing process for liposomal preparations of the present invention.

Materials and Methods:

In the example detailed below, DOPC, cholesterol, and tetramyristoyl cardiolipin were obtained from Avanti Polar Lipids, Inc., Alabaster, Ala. Paclitaxel was obtained from Hande Tech, Austin, Tex.; t-butanol and ethanol from J. T. Baker; sucrose from Mallinckrodt; and D-alpha tocopheryl acid succinate from Sigma.

Liposome Size measurements were made using Partcile Sizing Systems (PSS, CA) Z-380 instrument. Lyophilization was carried out using Genesis 25-EL (manufactured by VirTis). Pellicon 2 Tangential Flow Filtration system and the 100 kD MWCO polyether sulfone membrane cassettes were obtained from Millipore Corporation, Bedford, Mass.

For freeze fracture electron microscopy, the sample was quenched using a sandwich technique in liquid nitrogen cooled propane at a cooling rate of 10,000 Kelvin per second to avoid ice-crystal formation and artifacts during cryo-fixation process. The cryo-fixed sample was fractured using a JEOL-JED-9000 freeze etching equipment and the exposed fracture planes were shadowed with platinum for 30 seconds at an angle of 25-35° and coated with carbon for 35 sec. The replicas were cleaned and examined using Philips CM 10 electron microscope.

Preparation of Liposomal Paclitaxel Formulation with t-butanol Solvent by the Inventive Method:

200 ml and 500 ml batches of liposomal paclitaxel formulation at 1 mg/ml of the active principal were prepared as described below by the inventive method. For the purpose of this example, the process is described in detail with a 200 ml batch as an example.

Table 1 lists the formulation composition and the batch quantities used in the preparation.

TABLE 1
Formulation composition and batch quantities for liposome
based paclitaxel formulation using t-butanol

| | Quantity * | Batch |
|---|---|---|
| Chemical | (mg/ml) | Quantity |
| DOPC | 27.00 | 5.40 g |
| Cholesterol | 0.75 | 0.15 g |
| Tetramyristoyl | 2.45 | 4.90 g |
| Cardiolipin | | |
| Tocopheryl acid | 0.31 | 0.06 g |
| succinate | | |
| Paclitaxel | 1.0 | 0.20 g |
| t-Butanol ** | 0.05 ml | 8.0 g |
| 10% Sucrose solution in | Q.S. to 1.04 g | 208 g |
| normal saline | | |

* Final intended concentration of the ingredients in the formulation
** Specific gravity 0.789 g/mL. To be removed during the process

8.0 g of t-butanol solvent (after maintaining the solvent container at 35-40° C.) was first weighed in to pre-tared beaker with a stir bar.

200 mg of paclitaxel was weighed and added to t-butanol with mixing while maintaining the solvent temperature above 35° C. by heating on a hotplate. The beaker was covered with an aluminum foil to prevent evaporative loss of solvent.

After paclitaxel was completely dissolved (duration about 15-20 min), 150 mg of cholesterol was weighed separately and added to t-butanol solution containing paclitaxel and mixed until completely dissolved (duration 3-5 minutes).

490 mg of tetramyristoyl cardiolipin, 5.4 g of DOPC, and 62 mg of tocopheryl acid succinate were weighed individually and added in that order to t-butanol solution and mixed until the solution was free of any undissolved lipid while maintaining the solution temperature between about 40-50° C. Total duration for dissolution of all added components into solution was about 45 min, and the solution temperature was about 48° C.

The aqueous phase solution of 10% sucrose and 0.9% sodium chloride (4000 ml) was prepared by dissolving 400 g of sucrose and 36 g of sodium chloride in deionized water (Milli Q systems) and the solution was filtered through a MilliPak 20 sterilizing filter.

190 g of the filtered sucrose solution was weighed into a pre-tared jacketed glass container and fitted with a circulating water bath set at 36° C. to maintain the temperature.

The sucrose solution was mixed using a Labmaster Lightnin mixer at 300 rpm for 10 minutes to equilibrate the solution temperature to 35° C.

T-butanol solution containing paclitaxel and the lipid fraction were added to the aqueous solution with mixing at 300 rpm in a steady stream in one minute. The weight of lipid fraction (as solution) added was about 15 g.

The resulting solution, immediately upon completion of t-butanol solution addition was turbid with slight translucence which is characteristic of liposomes. The temperature of bulk liposome solution immediately after formation was measured to be 36° C. and the solution was mixed for an additional 10 minutes at 300 rpm. The mixing speed was increased to 500 rpm for an additional 30 minutes while the bulk liposomes were cooled to 25° C.

Liposome size measurement of bulk liposomes showed that they are multi lamellar liposomes with a mean size of 1.3 microns. The pH of bulk liposomes was measured to be 4.63.

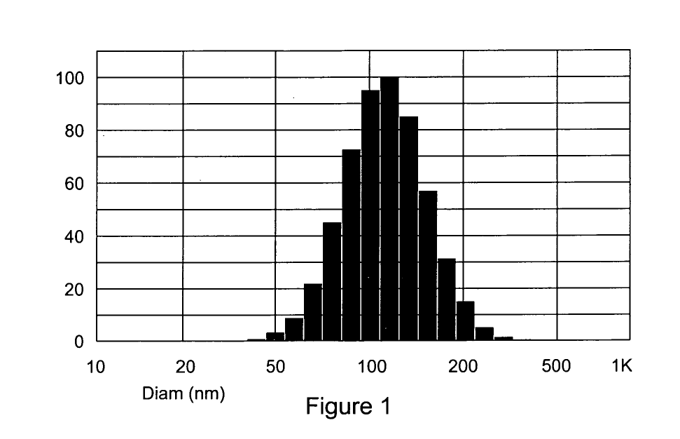

The bulk liposomes were size reduced by extrusion through 0.2μ and 0.1μ pore size polycarbonate membrane filters at 100-200 psi pressure. No drug precipitation was noted during the size reduction process on the filters establishing that the active principal, paclitaxel, is entrapped in the liposomes. Table 2 shows particle size data for liposome based paclitaxel after size reduction. FIG. 1 shows the size distribution of size reduced liposomes as measured by dynamic light scattering using PSS instrument. The mean diameter was measured at 120.7 nm (standard deviation=37.3 nm), and the distribution was as follows: 25%<86.8 nm, 50%<107.2 nm, 75%<132.3 nm, 90%<158.8 nm, and 99%<219.7 nm.

TABLE 2
Liposome size data for bulk liposomes during and after size reduction

| | Measured Size | | | |
|---|---|---|---|---|
| | | Standard | | |
| Process Step | Mean | deviation | D99 | Chi² |
| Bulk Liposomes | 1304 nm | 842 nm | 4388 nm | 121* |
| After 0.2 μ extrusion | 188.4 nm | 65.2 nm | 372.0 nm | 1.44 |
| After 0.1 μ extrusion | 120.7 nm | 37.3 nm | 219.7 nm | 2.55 |

*Chi² is too large. Nicomp distribution shows that majoirty ofliposomes are larger than 1 μ (1000 nm)

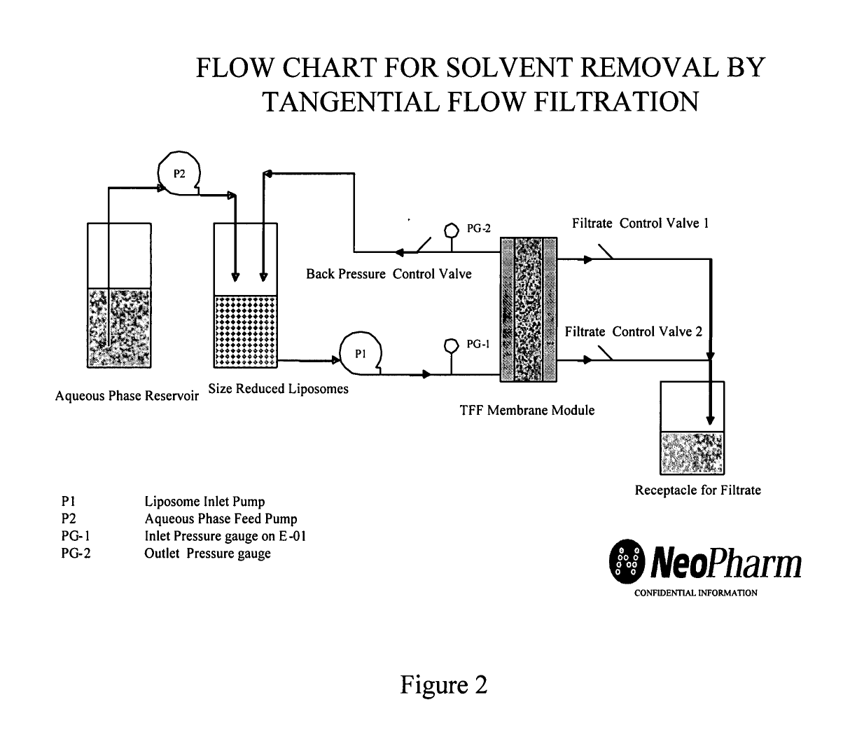

After size reduction, the liposomes were subjected to t-butanol solvent removal using Tangential flow-filtration (TFF) procedure. For this purpose, Pellicon 2 TFF system (Millipore Corp. Bedford, Mass.) assembled with a 0.1 square meter surface area polyether sulfone (PES) membrane cassette was used. Schematic representation of the TFF system used for freeing the bulk liposomes or size reduced liposomes of t-butanol employed in their formation is shown in FIG. 2. The specific membrane cassette used is fabricated with restricted channel screen (type C) capable of retaining any solute molecules (e.g., protein) or organized structures such as liposomes larger than 100,000 Daltons (molecular weight cut-off or MWCO 100 kD) allowing smaller solute molecules (such as sucrose and t-butanol) to pass through the membrane. Size reduced liposomes were first diluted two-fold (2×) by addition of about 200 g of 10% sucrose solution before they are introduced into the TFF system for solvent removal. The inlet flow, inlet pressure, outlet pressure (or back pressure), and filtrate flow were monitored during the process and represented in Table 3. A total of seven iterations (seven volumes of filtrate collected) were performed in concentration-dilution mode of operation.

TABLE 3
Tangential flow filtration process data for t-butanol
solvent removal from liposomes

| | Inlet | | Wt. of Filtrate | |
|---|---|---|---|---|
| Liposome Feed | Pressure (psi) | | Collected (in | Elapsed Time (in |
| Rate (ml/min) | Start | End | grams) | minutes) |
| 400 ml/min | 10 | 11 | 137 g | 5 min |
| 400 ml/min | 9 | 10 | 209 g | 6 min |
| 400 ml/min | 8 | 9 | 215 g | 7 min |
| 400 ml/min | 9 | 9 | 202 g | 7 min |
| 400 ml/min | 8 | 8 | 236 g | 8 min |
| 400 ml/min | 8 | 8 | 211 g | 8 min |
| 400 ml/min | 8 | 9 | 228 g | 10 min |

Solvent removal by tangential flow-filtration process in the case of 500 ml batch was performed in both concentration-dilution mode as well as continuous infusion of aqueous phase as t-butanol containing aqueous phase is removed as filtrate (feed and bleed mode) in two separate experiments. Liposome feed rates up to 600 ml/min were used which generated higher inlet pressures of up to 20 psi. Large scale process for solvent removal may use up to 100 l/min flow rates that generate inlet pressures of up to 50 psi. TFF membrane cassettes of similar (100 kD), smaller (10 kD) or larger (300 kD) MWCO with larger surface areas (up to 1000 sq. meter) can be used in commercial scale manufacturing to remove t-butanol. However, 100 kD MWCO membrane cassette is the preferred size to be used for the purpose of removing t-butanol from the liposomes. While larger MWCO membrane cassettes such as 300 kD and 500 kD can perform this function, some liposomes will also be lost to the filtrate in the process. The flow chart in FIG. 2 can be used for removal of water-miscible organic solvents (t-butanol and ethanol) used in bulk liposome formation from 200 ml scale batches to 200 l scale. While concentration-dilution mode described in the example is practical on small scale (up to 1000 ml), continuous mode (feed and bleed) is practiced on large scale.

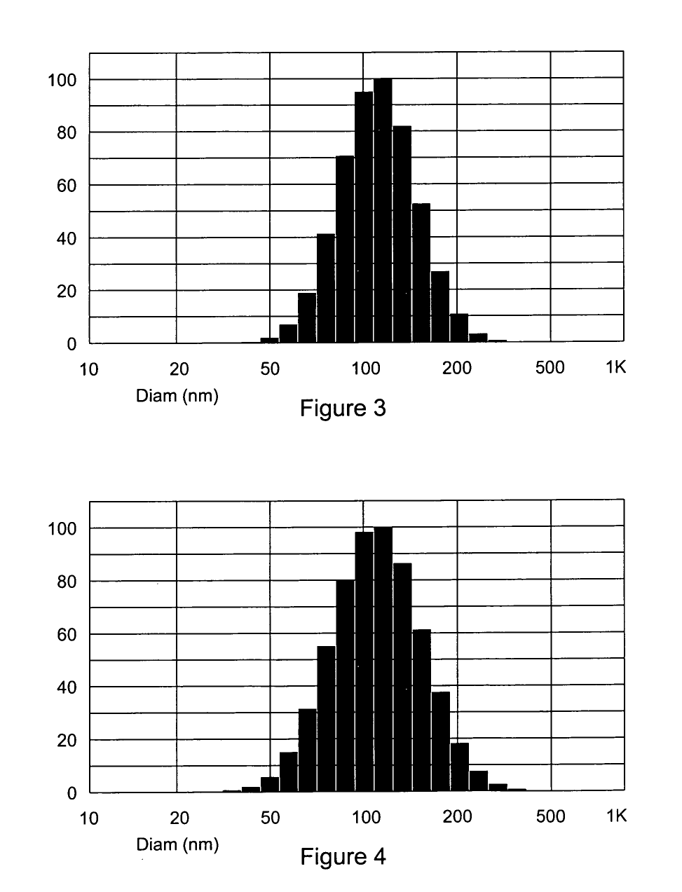

Particle size measurement of liposomes after the solvent removal by TFF process showed that the liposome size was not affected during the solvent removal process (see FIG. 3). The mean diameter was measured at 115.6 nm (standard deviation=33.6 nm), and the distribution was as follows: 25%<84.6 nm, 50%<103.2 nm, 75%<125.9 nm, 90%<150.2 nm, and 99%<202.6 nm.

Following solvent removal the liposomes were sterile filtered through a MilliPak 20 sterilizing filter before freeze-drying. Sterile filtered liposomes were filled in 20 ml glass (10.5 ml per vial) and freeze dried. Freeze dried liposomes were reconstituted with 10 ml of deionized water. Reconstituted liposomes were analyzed for liposome size, paclitaxel, DOPC, cholesterol, and cardiolipin contents. The size distribution of paclitaxel containing liposomes reconstituted after freeze drying (see FIG. 4) did not show any significant changes indicating that liposome integrity is preserved during the freeze drying process. The mean diameter was measured at 117.6 nm (standard deviation=40.2 nm), and the distribution was as follows: 25%<81.7 nm, 50%<103.3 nm, 75%<130.3 nm, 90%<160.4 nm, and 99%<230.5 nm.

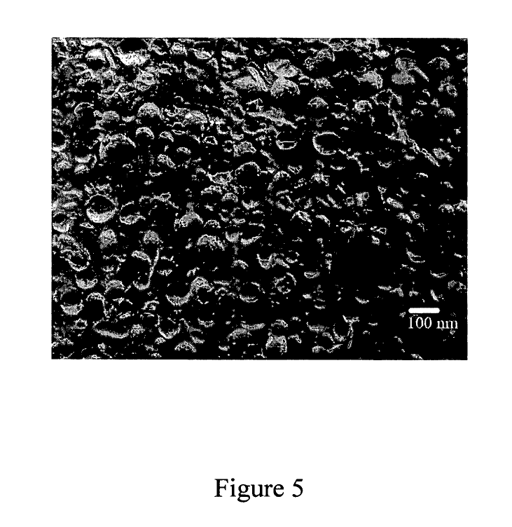

These liposomes were also assessed by freeze fracture electron microscopy, using the procedure described above. The electron micrographs obtained show uniform distribution of mostly spherical liposomes of single bilayer (also called small unilamellar vesicles or SUVs for short) with a diameter ranging from 20 to 150 nm. Major composition of the liposomes are individual and not associated or aggregated (see FIG. 5).

The results from analysis of bulk liposomes, after size reduction, sterile-filtration after solvent removal (Table 4) establish that the inventive method can be employed for manufacturing liposomes containing water in-soluble active principals, such as paclitaxel, using t-butanol.

TABLE 4
Paclitaxel and lipid fraction contents, liposome size results for
liposome based paclitaxel formulation prepared using t-butanol

| | | | | | Liposome Size | |
|---|---|---|---|---|---|---|
| | Paclitaxel | DOPC | Cholesterol | Cardiolipin | Mean | |
| Process Stage | mg/ml | mg/ml | mg/ml | mg/ml | (nm) | D99 (nm) |
| Target | 1.0 | 27.0 | 0.75 | 2.45 | 100-160 | 180-250 |
| Concentration/ | | | | | | |
| Liposme Size | | | | | | |
| Bulk | 1.04 | 27.5 | 0.74 | 2.11 | Not | Not |
| Liposomes | | | | | applicable | applicable |
| After Size | 1.02 | 27.5 | 0.74 | 2.26 | 120.7 | 219.7 |
| reduction | | | | | | |
| After solvent | 1.05 | 30.0 | 0.82 | 2.18 | 115.6 | 202.6 |
| removal and | | | | | | |
| before freeze | | | | | | |
| drying | | | | | | |
| After | 1.05 | 29.2 | 0.79 | 2.34 | 117.6 | 230.5 |
| reconstitution | | | | | | |
| of freeze-dried | | | | | | |
| liposomes | | | | | | |

All of the references cited herein, including patents, patent applications, and publications, are hereby incorporated in their entireties by reference.

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

What is claimed is:

1. A method of manufacturing a liposomal preparation, said method comprising:
(a) dissolving a lipid fraction in an organic solvent comprising t-butanol,
(b) adding the organic solvent comprising said lipid fraction to an aqueous solution,
(c) mixing the organic solvent comprising said lipid fraction with the aqueous solution at a temperature between about 25° C. and about 40° C. to form a bulk liposome preparation.

2. A method of manufacturing a liposomal preparation, said method comprising:
(a) dissolving a lipid fraction in an organic solvent comprising t-butanol and ethanol,
(b) adding the organic solvent comprising said lipid fraction to an aqueous solution,
(c) mixing the organic solvent comprising said lipid fraction with the aqueous solution at a temperature between about 25° C. and about 40° C. to form a bulk liposome preparation.

3. The method of claim 1 or 2, wherein the lipid fraction comprises one or more lipids selected from a group consisting of cholesterol, dioleoylphosphatidylcholine (DOPC), tetramyristoyl cardiolipin, tocopheryl acid succinate, and 1,3-Bis-(1,2-bis-tetradecyloxy-propyl-3-demethylethoxyammoniumbromide)-propan-2-ol [(R)-PCL-2].

4. The method of claim 3, wherein the lipid fraction consists of cholesterol, dioleoylphosphatidylcholine (DOPC), tetramyristoyl cardiolipin, and tocopheryl acid succinate.

5. The method of claim 4, wherein the lipid fraction is formed by sequential addition of cholesterol, dioleoylphosphatidylcholine (DOPC), tetramyristoyl cardiolipin and tocopheryl acid succinate.

6. The method of claim 3, wherein DOPC comprises a majority of the lipids.

7. The method of claim 1 or 2, wherein the lipid fraction is dissolved in the organic solvent at a temperature between about 35° C. and about 65° C.

8. The method of claim 7, wherein the temperature is between about 45° C. and about 55° C.

9. The method of claim 1 or 2, wherein the aqueous solution is at least 90 percent of the bulk liposome preparation.

10. The method of claim 1 or 2, wherein the aqueous solution further comprises one or more tonicity adjusters.

11. The method of claim 1 or 2, wherein the aqueous solution further comprises one or more protective sugars.

12. The method of claim 1 or 2, wherein the organic solvent comprising said lipid fraction is mixed with said aqueous solution at a temperature between about 30° C. and about 40° C.

13. The method of claim 12, wherein the temperature is between about 30° C. and about 35° C.

14. The method of claim 1 or 2, wherein the organic solvent comprising the lipid fraction is mixed with the aqueous solution at a speed of between about 200 rpm and about 1000 rpm.

15. The method of claim 14, wherein the organic solvent comprising the lipid fraction is mixed with the aqueous solution at a speed of between about 500 rpm and about 1000 rpm.

16. The method of claim 15, wherein the organic solvent comprising the lipid fraction is mixed with the aqueous solution at a speed of between about 600 rpm and about 800 rpm.

17. The method of claim 1 or 2, wherein the organic solvent comprising said lipid fraction is added to the aqueous solution for a duration of between about 5 minutes and about 1 hour.

18. The method of claim 17, wherein the duration is between about 10 minutes and about 45 minutes.

19. The method of claim 18, wherein the duration is between about 15 minutes and about 30 minutes.

20. The method of claim 1 or 2, further comprising the addition of one or more active principals.

21. The method of claim 20, wherein one or more active principals are water-soluble active principals.

22. The method of claim 20, wherein one or more active principals are added after removal of the organic solvent.

23. The method of claim 20, wherein one or more active principals are added before bulk liposome formation.

24. The method of claim 23, wherein one or more active principals are added to said organic solvent.

25. The method of claim 24, wherein one or more active principals are added to the organic solvent prior to the addition of the lipid fraction.

26. The method of claim 24, wherein one or more active principals are added to the organic solvent after the addition of the lipid fraction.

27. The method of claim 24, wherein one or more active principals are added to the organic solvent at a temperature above 35° C.

28. The method of claim 27, wherein one or more active principals are added to the organic solvent at a temperature between about 35° C. and about 65° C.

29. The method of claim 28, wherein one or more active principals are added to the organic solvent at a temperature between about 40° C. and about 55° C.

30. The method of claim 23, wherein one or more active principals are added to the aqueous solution.

31. The method of claim 30, wherein one or more active principals are added to the aqueous solution prior to addition of the organic solvent.

32. The method of claim 20, wherein the organic solvent comprising said lipid fraction is mixed with said aqueous solution at a rate to form liposomes without precipitation of the one or more active principals.

33. The method of claim 20, wherein one or more active principals are selected from a group consisting of anticancer agents, antisense oligonucleotides and antifungal agents.

34. The method of claim 33, wherein the anticancer agent is selected from a group consisting of taxane, mitoxantrone, camptothecin, doxorubicin, daunorubicin, methotrexate, adriamycin, tamoxifen, toremifene, cisplatin, epirubicin, gemcitabine HCl, and derivatives thereof.

35. The method of claim 34, wherein the taxane is paclitaxel.

36. The method of claim 33, wherein the antisense oligonucleotide is directed to an oncogene.

37. The method of claim 36, wherein the oncogene is raf.

38. The method of claim 1 or 2, further comprising size reducing the bulk liposome preparation to obtain a size-reduced liposomal preparation.

39. The method of claim 38, wherein the size reduction is achieved without precipitation.

40. The method of claim 38, wherein said size reduction is achieved by extrusion of the bulk liposome preparation through polycarbonate filters.

41. The method of claim 40, wherein the filters are between about 0.2 μm and about 0.1 μm.

42. The method of claim 38, wherein size reduction is achieved by extrusion of the bulk liposome preparation at pressures between about 200 psi and about 800 psi.

43. The method of claim 1 or 2, further comprising substantially removing the organic solvent.

44. The method of claim 43, wherein the organic solvent is substantially removed by diafiltration using a tangential flow filtration process.

45. The method of claim 1 or 2, further comprising sterile filtering said liposome preparation.

46. The method of claim 1 or 2, further comprising freeze drying said liposome preparation.


SEQUENCE LISTING (1 sequence)

SEQ ID NO: 1 - DNA, 15 bp, Artificial
  Synthetic
gtgctccattgatgc